(19) United States
(12) Patent Application Publication
(10) Pub. No.: US 2007/0065803 A1
(43) Pub. Date: Mar. 22, 2007

(54) Patterns for a skeletal system

(75) Inventors: Pierre Jurdic, Lyon (FR); Olivier Destaing, Lyon (FR); Frederic Saltel, Lyon (FR); Edith Anne-Marie Bonnelye, Lyon (FR)

Correspondence Address: BUCHANAN, INGERSOLL & ROONEY PC, POST OFFICE BOX 1404, ALEXANDRIA, VA, 22313-1404, US

(73) Assignees: CENTRE NATIONAL DE LA RECHERCHE SCIENTIFIQUE, PARIS (FR); ECOLE NORMALE SUPERIEURE DE LYON, LYON (FR)

(21) Appl. No.: 10/559,819

(22) PCT Filed: Jun. 11, 2004
(86) PCT No.: PCT/FR04/01470

§ 371 Date: Jun. 2, 2006

(30) Foreign Application Priority Data
Jun. 11, 2003 (FR) 03 07025
Mar. 23, 2004 (FR) 04 02993

(51) Int. Cl. A61F 2/28; C12N 5/08; C12Q 1/00

(52) U.S. Cl. 435/4; 623/23.63; 435/366; 435/325

(57) ABSTRACT

The present invention relates to a bone system model comprising a mineralized matrix and osteoblasts, chararacterized in that the osteoblasts are deposited onto the matrix so as to form a layer at confluence and/or nodules, and the osteoclasts are deposited onto said layer and/or said nodules, and also to bone system models affected by various pathologies. The invention also covers the uses for carrying out screenings and tests for aggressiveness and for toxicity.

The present invention relates to bone system models.

It relates more particularly to in vitro bone system models comprising a resorbable matrix, osteoclasts and osteoblasts, to a method of selecting the matrices that can be used for the models according to the invention, and also to bone system models that mimic a particular pathology.

Bone consists of cells and of a extracellular matrix which is mineralized. The cell population is composed of two cell types: osteoclasts which degrade the bone matrix and osteoblasts which reconstruct it. Up until now, the majority of research studies carried out on the subject have been directed toward the specific study of osteoclasts, as bone cells responsible for degradation of the bone matrix, toward the specific study of osteoblasts, or toward the choice of artificial matrices capable of mimicking the human bone matrix. In particular, the article by Shibutani et al. (J Biomed Mater Res, Use of glass slides coated with apatite-collagen complexes for measurement of osteoclastic resorption activity, 31:43-49, 1996) describes an example of a collagen-based mineralized matrix.

More recently, the influence of the size of hydroxypatite powder particles on osteoclast activity was studied in an article by Sun et al. (J Biomed Mater Res, The influence of hyroxypatite particles on osteoclast cell activities, 45(4): 311-21, 1999). A coculture of osteoblast and osteoclast cells is particularly described therein.

The inventors' studies have led them to develop a bone system model comprising a mineralized matrix and osteoclasts, characterized in that osteoblasts are deposited onto the matrix so as to form a layer at confluence and/or nodules, the osteoclasts being deposited onto said layer and/or said nodules.

The arrangement of the two cell types is particularly important for reconstituting this bone system serving as a model. This is because human bone cells are only activated in a certain environment. The inventors have succeeded in reconstituting this environment by preparing a layer of osteoblasts at confluence or osteoblast nodules, and placing osteoclasts on this layer or these nodules. Unexpectedly, the inventors have noted that the osteoclasts, cells that are approximately ten times larger than the osteoblasts, are capable of making their way through the joint population of osteoblasts (in the form of a layer at confluence or of nodules) in order to proceed to exert their resorption activity directly on the bone matrix. The osteoclasts are then located under the population of osteoblasts, after approximately the 2nd hour following deposition of the osteoclasts.

In order to verify that the migration of the osteoclasts through the osteoblast layer is indeed a bone tissue-specific mechanism, the inventors repeated the experiment in an identical manner on a cover slip of dentine (support closest to bone). Now, on this support, the migration through the osteoblast layer was also observed.

Such an observation therefore makes it possible to validate this model as appropriate for mimicking the bone system and more particularly the murine or human bone system. Other observations, described in the examples of the present application, confirm this validation.

Advantageously, the bone system model according to the invention comprises a matrix composed of collagen and of calcium phosphate and/or calcium phosphate derivatives. Preferably, the calcium phosphate derivative is hydroxyapatite.

Preferably, the ratio of osteoclasts to osteoblasts is approximately 1/10 to 1/25. This ratio is variable since it depends on the phenomenon that it is desired to observe. Specifically, if too great a number of osteoclasts is added, there will be too great and too rapid a degradation of the matrix, thus impairing any possible quantification of the material resorbed (measurement of the surface that is no longer mineralized).

The model according to the invention provides the means for testing the effectiveness of known or novel drugs with the prospect of developing novel therapeutic treatments in a normal or pathological bone context.

The term “drugs” is intended to mean biologically active molecules.

More particularly, the invention makes it possible to test the potential of any drugs that are already known (for example: biphosphonate, PTH, vitamin D, etc.), or novel drugs on bone formation (osteoblasts) and/or on bone invasion and/or migration and/or resorption (osteoclasts), thus generating a rapid in vitro test for evaluating the therapeutic potential of any molecules that can act on bone metabolism, but also the harmful effects (side effects) of any drugs used for other pathologies that do not affect bone (for example: diabetes, cardiac diseases, etc.).

In the application, the term “migration” used alone signifies the moment of the osteoclasts during resorption. Similarly, the term “invasion” used alone refers to the colonization of the support to be resorbed. On the other hand, when these terms are used to refer to the crossing of the osteoblast layer, they are followed by an expression specifying this.

According to one embodiment, the osteoblasts and/or osteoclasts deposited can be genetically modified. The depositing of genetically modified cells makes it possible to study the behavior of these cells and the evolution of the bone system, more particularly with a view to a gene therapy. The use of these genetically modified cells is particularly suitable on the bone model systems exhibiting a pathology, as subsequently described.

The present invention is also directed toward a method of selecting matrices for reconstituting a bone system model, characterized in that a mineralized matrix is subjected to the following process:
depositing of a layer and/or of nodules of osteoblasts at confluence onto the matrix,
depositing of isolated osteoclasts onto the layer and/or the nodules,
observation of the invasion of the osteoclasts through the layer and/or the nodules of osteoblasts,
observation of the resorption of the mineralized matrix,
selection of the matrices on which the osteoclasts are located between the matrix and the layer and/or the nodules of osteoblasts and on which a resorption is observed;
   and also the artificial matrices selected using said method.

In fact, the behavior of the cells on the matrix makes it possible to determine whether the environment chosen for mimicking the bone system is suitable. By observing the cell behavior, it is therefore possible to determine whether the matrix chosen is a good bone matrix model.

Advantageously, the material of the matrix to be tested may be chosen from all biomaterials, i.e. materials compatible with living tissue. A modification of the matrix (addition of various protein compounds or other compounds) may lead to the development of novel biomaterials.

As mentioned above, the invention also provides bone system models that mimic bone pathology. These models are preferably prepared from cells (osteoblasts and/or osteoclasts) extracted from tissues originating from any bone pathologies.

In particular, the invention provides a bone system model that is cancerous, affected by osteoporosis, affected by osteomalacia and/or affected by rheumatoid arthritis.

The expression “bone system model which is cancerous” is intended to mean the bone system models corresponding to the following pathologies:
a disease where there is a primary cancerous tumor,
a disease where there is a primary cancerous tumor (breast, prostate, etc.) with metastases,
a disease where there is a bone cancer.

These models mimic pathologies using the bone system model described above, but bearing a certain number of modifications.

For example, for the bone system model that is cancerous, the modifications are as follows:
the osteoblasts and/or the osteoclasts are derived from normal, ovariectomized and/or orchidectomizd animals, etc., and
cells derived from cancer cell lines are also deposited.

To study a bone cancer, the cells deposited will, for example, be derived from a bone cancer cell line. In the other cases, cells derived from a cell line of a primary tumor that may (breast, prostate, etc.) or may not have a metastatic potential will be deposited.

This model makes it possible to observe the phenomena of bone tissue colonization by tumor cells and to visualize the metastatic phase. This is because, as shown by the invasion of the osteoclasts through the osteoblast layer, the cells placed in an environment mimicking the bone system have the ability to move in this environment.

This model is more particularly suitable for carrying out a test for the aggressiveness of tumor cells (see example 4). The existance of many primary cancers (breast, prostate, etc.) capable of metastasizing to bone is now an established fact in cancerology. Now, this bone cancer that ensues therefrom proves, in the majority of cases, to be incurable. The invention makes it possible to test, in vitro, the aggressiveness (invasion, migration, proliferation) of tumor cells (for example, derived from breast tumors or prostate tumors) originating from patient biopsies. These tumor cells are deposited onto the bone system model according to the invention and thus make it possible to estimate the aggressive potential of the cells of the primary tumor in terms of their invasive capacity, migratory capacity and/or proliferative capacity, and also to establish a prognosis with regard to the development of a secondary bone cancer.

More specificaly, the invention provides a rapid in vitro test for evaluating the therapeutic potential (chemotherapy and/or radiotherapy) of all molecules that can be used in cancerology, in order to reduce the appearance and/or to contribute to the anticancer treatment of a bone cancer.

The bone system model affected by osteoporosis comprises the following modifications:
the osteoblasts and/or the osteoclasts are derived from normal, ovariectomized and/or orchidectomized animals, the osteoporosis then being induced chemically in situ, and/or from knock-out animals which are transgenic for any molecules for which the modulation of expression induces a decrease in bone mass,
while the bone system model affected by osteomalacia is modified as follows:
   the osteoblasts and/or the osteoclasts are derived from normal animals, the osteomalacia then being induced chemically in situ, and/or animals that are knock-out for the vitamin D receptor or for any other molecules capable of inducing osteomalacia.

Many molecules are known to induce osteoporosis. By way of example, mention may be made of dexamethasone, hydrocortisone, prednisolone and its derivatives, fluocortolone, calcium heparin or sodium heparin.

Similarly, osteomalacia can be induced by the following molecules:
aluminum salts, barbital and its derivatives, retinol and beta carotene.

Finally, the bone system model affected by rheumatoid arthritis can comprise the following modifications:
the osteoblasts and/or the osteoclasts are derived from normal animals, the rheumatoid arthritis then being induced chemically in situ, and/or from knock-out animals which are transgenic for any molecules capable of inducing rheumatoid arthritis or from animals having been given injections of collagen type II, or of any other substances capable of inducing an articular inflammation mimicking rheumatoid arthritis.

The molecules that can induce rheumatoid arthritis are, by way of example, certain α-interferons, certain vaccines (BCG, hepatitis B, rubella, etc.), cortivazol, certain lithium salts, and ampicillin.

The present application also protects the use of the various models for carrying out screenings for therapeutic molecules and effectiveness tests. Using the models according to the invention, the effectiveness of the various models currently known and of those that will be updated may be compared. By way of example, the effectiveness test for osteoporosis will be able to compare molecules known to reestablish bone mass, such as biphosphonate, estrogens and all the novel molecules updated by means of the model used to carry out a screening. The effectiveness test for osteomalacia will be able to take vitamin D as reference molecule, while the test for rheumatoid arthritis will be able to take aspirin and/or the non-steroidal anti-inflammatories as reference molecules.

Advantageously, the models according to the invention are particularly suitable for carrying out tests for the toxicity of a chemical compound, in which at least one concentration of said compound is tested on a model according to the invention. Such tests make it possible to evaluate the side effects of medicinal products on bone physiology (for example, medicinal products for diabetes, antibiotics), the toxic effects of pollutants (dioxin, insecticides, etc.), etc. Advantageously, several concentrations will be tested in order to establish a relationship between the concentration of the compound and the side effects engendered in the bone system model.

A bone system model affected by osteomyelitis and/or by a bone infection can also be constructed by adding into the model system according to the invention various bacterial or viral strains. By way of example, mention will be made of the following strains: Enterobacter cloacae, staphylococcus aureus, beta-hemolytic streptococcus A, Haemophilus influenzae type b, salmonellae, Pseudomonas and/or pneumococci, etc.

Other characteristics and advantages of the invention will appear in the examples that follow, with references to the figures, which represent, respectively:

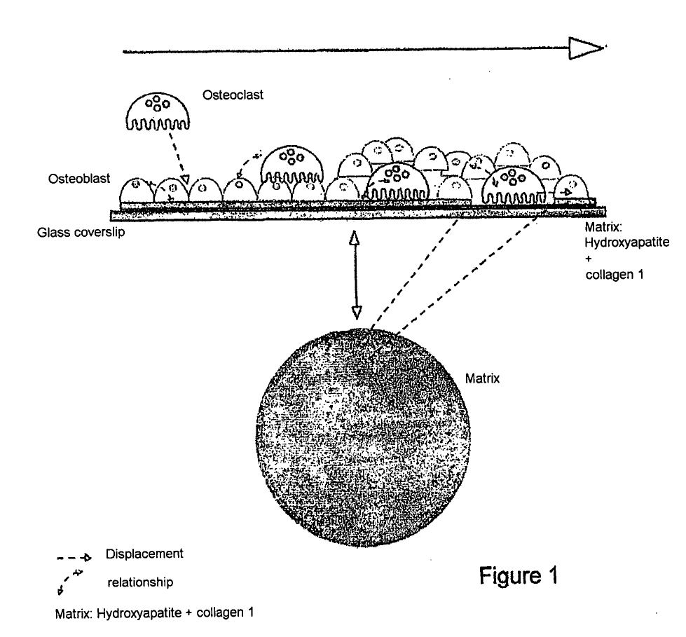

FIG. 1, a diagram of the process of crossing of the osteoblast layer by the osteoclasts and of osteoclastic resorption,

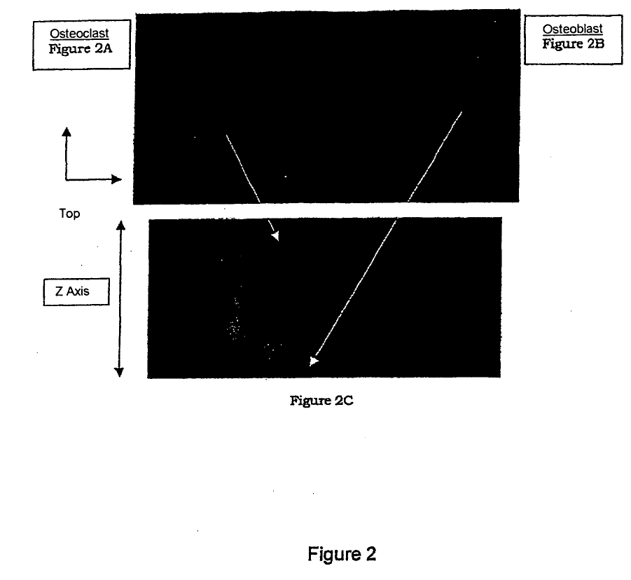

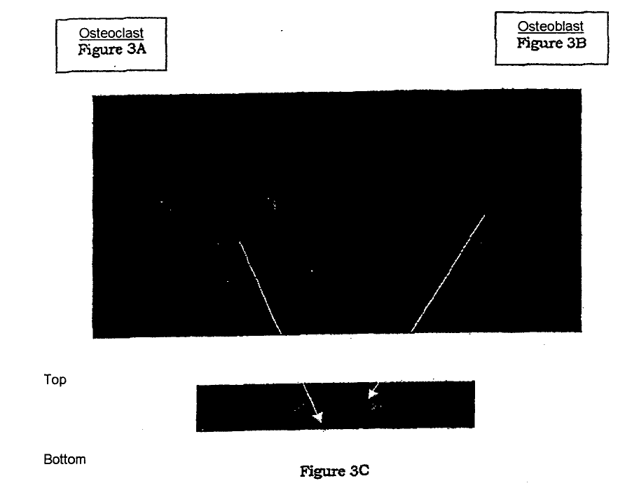

FIGS. 2 and 3, images obtained by confocal microscopy 3D analysis, showing the organization of the two cell types, A: osteoclast, B: osteoblast, C: section along Z, and,

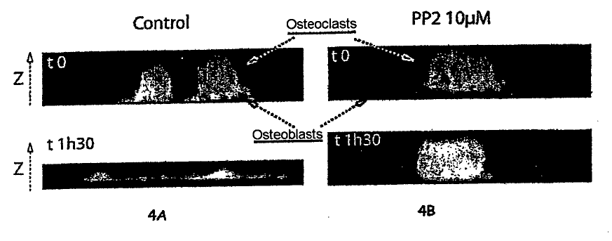

FIG. 4, images obtained by confocal microscopy 3D analysis, showing the organization of the two cell types according to the section along Z, in order to determine the effects of PP2 (FIG. 4B) in comparison with the control trial (FIG. 4A).

### EXAMPLE 1

### Reconstitution of a Celluarized Bone Interface for a Resorption Test—Layer of Osteoblasts at Confluence

a. Matrix

The mineralized matrix is prepared either on glass cover slips (for microscopy) or on plastic treated for cell culture. The support is first covered, for 15 h, with a solution of collagen I 0.1 mg/ml diluted in 0.1M acetic acid. The excess collagen is removed and the support is then covered, for one week at 37° C., with a solution of 200 mM TRIS, pH8.5, 0.4/l of alkaline phosphatase, 0.4 g/l of phosvitin and 3 g/l of dimethyl suberimidate chloride.

This step is followed by the mineralization per se. This mineralization consists of two successive steps:
1- the support is covered with a solution of 200 mM TRIS, pH8.5, 0.4 g/l of alkaline phosphatase and 0.4 g/l of phosvitin for 3 h, then,
2- this solution is replaced with a solution of 200 mM TRIS, pH8.5, and of 126.06 g/l of β-glycerophosphate for 20 h.

These two steps are repeated 10 times. The network of collagen I can be supplemented with other proteins (osteopontin, vitronectin, BSP, osteocalcin, collagen type I conjugated to a fluorescent agent, for example rhodamine, etc.) or with other substitutes (for example, mineral substitutes: fluorine, strontium ranelate, etc.).

b. Osteoblasts

Cells of the murine osteoblast line MC 3T3 are placed in culture in α-MEM medium supplemented with 10% by volume of fetal calf serum, 10⁻⁸M dexamethasone and 0.028 mM ascorbic acid. The cells are then detached and seeded at confluence onto the mineralized support.

The osteoblasts may also be derived from rat osteoblast (Ros) or human osteoblast (HEPM, hFOB) lines and prepared according to the same protocol. Alternatively, a protocol for primary culture of osteoblasts is provided in example 2.

c. Osteoclasts

The osteoclasts derived from human or murine primary cultures or from lines (for example, the RAW line or the human line GCT23) are obtained after 7 days of differentiation as described in Destaing et al. (Mol Biol Cell, Podosomes display actin turnover and dynamic self-organization in osteoclasts expressing actin-green fluorescent protein, 14(2):407-16, 2003). The osteoclast precursors are cultured in the presence of α-MEM medium supplemented with 10% by volume of fetal calf serum and of two recombinant cytokines: M-CSF and RANK-L (20ng/l). The cells are placed at 37° C. and 5% of CO₂ and the medium is changed every two days for 7-8 days. The differentiated osteoclasts are detached with a solution of EDTA at 0.25 mM diluted in 1×PBS. The osteoclasts are seeded at a density of 10 cells/mm².

d. Fixing

The fixing is carried out in 1×PBS to which 3.7% formaldehyde has been added, for 10 mins.

e. Results

The resorption of the mineralized matrix is observed by photon microscopy from the 6th or 7th hour following assembly of the model (time elapsed between the first osteoclasts-osteoblasts contact and the resorption of the matrix).

A second experiment was carried out in order to demonstrate that the osteoclasts are capable of crossing the dense layer of osteoblast cells before forming resorption holes.

By confocal microscopy 3D analysis, it was possible to demonstrate the invasive capacity of the osteoclasts and to quantify the resorption by measuring the surface area of the substrate that is no longer mineralized.

The cells are fixed for one hour and four hours after the depositing of the osteoclasts.

FIG. 2 comprises three images showing the location of the various cells one hour after the depositing of the osteoclasts.

FIG. 2A is an image of the osteoclast. The latter exhibits strong polarity, which demonstrates the active state of the cells. In fact, an osteoclast deposited onto an unsuitable support exhibits a small thickness (non polarized). On the other hand, on a suitable support, said osteoclast thickens so as to form a basal pole (contact with the osteoblasts) and an apical pole (contact with the medium). The inventors observed that this polarity was maintained while the osteoblast layer was being crossed and was maintained during the resorption. However, the basal pole is in contact with the matrix, while the apical pole is in contact with the osteoblasts.

FIG. 2B is an image of the osteoblast. The presence of many actin filaments is observed. Actin stress fibers constitute a marker that is present in the MC3T3 osteoblasts.

FIG. 2C is a section along Z, that makes it possible to visualize the location along the Z axis of the two cells previously photographed. This image shows that the osteoclast is located above the osteoblast layer (continuous light line).

FIG. 3 also comprises three images corresponding to the three images in FIG. 2. FIG. 3A is a photograph of the osteoclast. It has changed shape, it has become flattened. FIG. 3B represents the osteoblast layer. It is at this time located above the osteoclast, the latter being in direct contact with the matrix (FIG. 3C), and exhibits the organization of an osteoclast in the process of resorption (the <<sealing zone >>, structure characteristic of the resorbing cells).

### EXAMPLE 2

### Reconstitution of a Celluarized Bone Interface for a Resorption Test—Osteoblast Nodules

a. Matrix

The matrix is prepared as described in example 1.

b. Osteoblasts

The osteoblasts used can be derived from human or murine primary cultures or from lines as described in example 1.

The cells were isolated by enzymatic digestion (collagenase (Sigma #C-0130)) from calvaria of 2-day old mice. The cells obtained from the last four digestion steps (population II-V) are subsequently seeded into T75 flasks in αMEM medium containing 15% of fetal calf serum (Flow Laboratories, McLean, Va.) and 100 μg/ml penicillin G (Sigma Chemical Co., St. Louis, Mo.), 50 μg/ml gentamycin (Sigma), and 0.3 μg/ml fungizone (Flow Laboratories). After incubation for 24 h, the adherent cells are rinsed with PBS, treated with trypsin (0.01%) in a citrate salt solution, resuspended in the standard medium described above and seeded onto twelve-well plates on the matrix described in example 1 at 10⁴ cells/well. After incubation for 24 h, the medium is changed and supplemented with ascorbic acid (50 μg/ml) and with sodium β-glycerophosphate (10 mM). The medium is subsequently changed every two days. All the plates are incubated at 37° C. under an atmosphere of 95% air and 5% CO₂.

A primary culture of human osteoblasts is also possible.

c. Osteoclasts

The osteoclasts are prepared according to the method described in example 1.

d. Fixing

The fixing is carried out as in example 1.

e. Results

The resorption of the mineralized matrix is observed by photon microscopy from the 7th hour following assembly of the model.

A second experiment was carried out in order to demonstrate that the osteoclasts are capable of crossing the osteoblast nodules before forming the resorption holes.

By confocal microscopy 3D analysis, it is possible to demonstrate the invasive capacity of the osteoclasts and to quantify the resorption by measuring the surface area of the substrate that is no longer mineralized.

As in the previous example, we were able to observe the crossing of the osteoblasts by the osteoclasts and also the resorption holes made by the osteoclasts.

EXAMPLE 3
Variants of the cell types deposited

| bone pathologies: | description of the system |
|---|---|
| osteoporosis (degradation of the | osteoblasts from ovariectomized |
| bone matrix due to the arrest | (female) or orchidectomized |
| of estrogen synthesis in women, | (male) mice + normal osteoclasts |
| of testosterone synthesis in men) | normal osteoblasts + osteoclasts |
| | from ovariectomized (OVX) or |
| | orchidectomized (ORX) mice |
| | osteoblasts + osteoclasts |
| | from OVX (female) or ORX (male) |
| | mice/normal osteoclasts + |
| | osteoblasts. |
| osteomalacia (vitamin D | osteoblasts from VDR (vitamin |
| deficiency engendering | D receptor) KO mice + |
| poor mineralization of | normal osteoclasts |
| the bone matrix) | normal osteoblasts + osteo- |
| | clasts from VDR (vitamin D |
| | receptor) KO mice |
| | osteoblasts + osteoclasts |
| | from VDR (vitamin D receptor) |
| | KO mice/normal osteoclasts + |
| | osteoblasts. |
| rheumatoid arthritis (RA) | osteoblasts from mice |
| (inflammation caused by an | (injection collagen type II |
| increase in bone resorption | capable of inducing RA in |
| by the osteoclasts) | mice in vivo) |
| | normal osteoclasts |
| | normal osteoblasts + |
| | osteoclasts from mice |
| | (injection collagen type II |
| | capable of inducing RA |
| | in mice in vivo) |
| Other bone pathologies and any | osteoblasts + osteoclasts |
| KOs already known to exhibit a | from RA mice/normal |
| deficiency in bone metabolism | osteoclasts + osteoblasts |
| (ALP, src, c-fos, OPG, Rankl, | |
| receptor for estrogens, | |
| aromatase, leptin, etc.) and | |
| which could be associated with | |
| human mutations not yet | |
| determined: and any new knock | |
| outs. | |

test for the effect of known drugs - Estrogens (SERM, tamoxifen,
raloxifen)- testosterone, PTH (Parathyroid hormone), prostaglandins
(PGE2), biphosphonates (alendronate), vitamins D, osteoprotegerin
(OPG), RANKL, NSAIDs (non-steroidal anti-inflammatory drugs, for
example: ibuprofen); glucocorticoids, T3 thyroid hormone, etc.
test for the effect of novel drugs

| cancerous pathologies | cells used |
|---|---|
| breast cancer: cancer which | normal osteoblasts and osteo- |
| metastasizes to bone tumor | clasts + breast cancer cell |
| ⅓ of metastatic | lines either aggressive (MDA- |
| osteosis and more than ⅔ | MB-231) or non-aggressive (MCF- |
| of female cancers | 7). Observation of colonization |
| | of mammary cells, in vitro |
| | formation of mammary tumors. |
| | abnormal (OVX) osteoblasts |
| | and osteoclasts + breast |
| | cancer cell lines. Test of the |
| | role of estrogens in the |
| | cancerization phenomenon |
| prostate cancer: cancer | normal osteoblasts and osteo- |
| which metastasizes to bone | clasts + prostate cancer cell |
| tumor | lines (LAPC-4). Observation of |
| one of the most common | colonization of prostate cells, |
| male cancers | in vitro formation of tumors |
| | abnormal (ORX) osteoblasts |
| | and osteoclasts + prostate |
| | cancer cell lines. Test of the |
| | role of testosterone in the |
| | cancerization phenomenon. |
| other cancers that can | human lung adenocarcinoma cell |
| metastasize to bone tumors: | line: A549 |
| lung cancer | human kidney carcinoma cell |
| kidney cancer | lines: ACHN, A704, Caki-1 |
| thyroid cancer | human thyroid carcinoma cell |
| digestive cancer | line: FRO |
| uterine cancer | human gastric carcinoma cell |
| ovarian cancer | line: EPG85-257P |
| | human endometrial carcinoma |
| | cell line: HEC-1 |
| | human epithelium ovarian |
| | cancer cell line: EOC |

test for the effect of drugs known to act on the decreasing of cell
invasion and proliferation: chemotherapeutic agents, cyclo-oxygenase
(COX) inhibitor, melatonin, etc.
test for novel drugs

### EXAMPLE 4

### Test for Tumor Cell Aggressiveness

One woman in 4 will die of breast cancer and one man in 3 will die of prostate cancer in France (EUCAN, cancer incidence mortality and prevalence in the EU 1996).

These two cancers are generally primary cancers which, when they are not stopped in time, metastasize to the bone, which cancers then prove to be irreparably fatal. The bone system models according to the invention should make it possible to study why these mammary cells or prostate cells metastasize virtually systematically to the bone.

The system according to the invention in fact makes it possible to visualize, in vitro, the invasion (or the chemotaxis of the products secreted by the cells of this cellularized bone interface) of mammary or prostate cancer cells into the bone matrix. Aggressive (MDA-MB-231) or non-aggressive (MCF-7) human breast cancer cell lines or alternatively human prostate cancer cell lines (LAPC-4) are deposited onto the system according to the invention. This simple experiment makes it possible to observe the behavior of these cells compared with other cell types known to metastasize very little to the bone, to observe the interactions between the tumor cells and the cells of the bone systems, or alternatively the formation of proliferative cell masses, and the possible degradation of the bone matrix.

Once the formation of tumors in vitro has been carried out, a second step consists in analyzing the effectiveness of anticancer drugs that are already known (chemotherapy, etc.) or alternatively in demonstrating, by screening, novel molecules that are active on cell proliferation (decrease in the cell mass or in the number of foci).

### EXAMPLE 5

### Screening for Molecules, for Therapeutic Purposes

An inhibitor of the Src tyrosine kinase, PP2, was tested in the model according to the invention. This product is known for its anti-resorptive properties and appears to constitute a potential therapeutic molecule for the treatment of osteoporosis. To prepare the bone interface according to the invention, osteoclasts derived from RAW-GFP were deposited onto a layer of MC3T3-type osteoblast cells. After a period of 1 hour to 1 hour 30 min, in the absence of treatment, the osteoclasts crossed this cell layer and spread out under the latter. On the other hand, if the osteoclasts are treated with the Src tyrosine kinase inhibitor PP2, at a concentration of 10 μM, the osteoclasts no longer cross the osteoblast cell layer (see FIG. 4).

PP2 is therefore capable of blocking the mechanisms of invasion of the osteoclasts in the model according to the invention, thus leading to a decrease in bone resorption (inability of the osteoclasts to reach the mineralized matrix).

This experiment makes it possible to demonstrate that the bone interface according to the invention can therefore be used for the purposes of screening for therapeutic molecules.

1. A bone system model comprising a mineralized matrix and osteoblasts, characterized in that the osteoblasts are deposited onto the matrix so as to form a layer at confluence and/or nodules, and wherein osteoclasts are deposited onto said layer and/or said nodules.

2. The bone system model as claimed in claim 1, characterized in that it mimics the human bone system.

3. The bone system model as claimed in claim 1, characterized in that the mineralized matrix is a matrix composed of collagen and calcium phosphate and/or calcium phosphate derivatives.

4. The bone system model as claimed in claim 1, characterized in that the mineralized matrix is a matrix composed of collagen and of hydroxyapatite.

5. The bone system model as claimed in claim 1, characterized in that the ratio of osteoclasts to osteoblasts is approximately 1/10 to 1/25.

6. The bone system model as claimed in claim 1, characterized in that the osteoblasts and/or osteoclasts deposited are genetically modified.

7. A method of selecting a matrix for reconstituting a bone system model, characterized in that a matrix is subjected to the following process:
depositing of a layer and/or nodules of osteoblasts at confluence onto the matrix,
depositing isolated osteoclasts onto the layer and/or the nodules,
observation of the invasion of the osteoclasts through the layer and/or the nodules of osteoblasts,
selection of the matrices on which the osteoclasts are located between the matrix and the layer and/or the nodules of osteoblasts.

8. The method of selection as claimed in claim 7, characterized in that it also comprises a step of observation of the resorption of the matrix, and in that the matrices on which a resorption is observed are selected.

9. Artificial matrices selected using the method as claimed in claim 7.

10. A bone system model that is cancerous, characterized in that the model as claimed in claim 1 is used, modified as follows:
The osteoblasts and/or the osteoclasts are derived from normal, ovariectomized and/or orchidectomized animals,
cells derived from cancer cell lines are also deposited.

11. A bone system model affected by osteoporosis, characterized in that the model as claimed in claim 1 is used, modified as follows:
the osteoblasts and/or the osteoclasts are derived from normal, ovariectomized and/or orchidectomized animals, the osteoporosis has been induced chemically in situ, and/or results from knock-out animals which are transgenic for any molecules for which the modulation of expression induces a decrease in bone mass.

12. A bone system model affected by osteomalacia, characterized in that the model as claimed in claim 1, is used, modified as follows:
the osteoblasts and/or the osteoclasts are derived from normal animals, and wherein the osteomalacia has been induced chemically in situ, and/or results from animals that are knock-out for the vitamin D receptor or for any other molecules capable of inducing osteomalacia.

13. A bone system model affected by rheumatoid arthritis, characterized in that the model as claimed in claim 1 is used, modified as follows:
the osteoblasts and/or the osteoclasts are derived from normal animals, the rheumatoid arthritis has been induced chemically in situ, and/or results from knock-out animals which are transgenic for any molecules capable of inducing rheumatoid arthritis or from animals having been given injections of collagen type II, or of any other substances capable of inducing an articular inflammation mimicking rheumatoid arthritis.

14. A bone system model affected by osteomyelitis, characterized in that the model as claimed in claim 1 is used, modified as follows:
at least one bacterial or viral strain chosen from Enterobacter cloacae, staphylococcus aureus, beta-hemolytic streptococcus A, Haemophilus influenzae type b, salmonallae, Pseudomonas, and/or pneumococci is added to the model system.

15. (canceled)

16. A test for tumor cell aggressiveness, characterized in that tumor cells are taken from a patient by biopsy, and the cells taken are deposited into a model as claimed in, claim 1 according to the pathological state of the patient, in order to observe the development of secondary bone cancer.

17. A test for the toxicity of a chemical compound, characterized in that at least one concentration of said compound is tested on a model as found in claim 1.
